(19) United States
(12) Patent Application Publication
(10) Pub. No.: US 2009/0275521 A1
(43) Pub. Date: Nov. 5, 2009

(54) Regulation of Microphage Migration Inhibitory Factor (MIF) Activity

(76) Inventors: Katherine Meyer-Siegler, Seminole, FL (US); Pedro Vera, West Largo, FL (US)

Correspondence Address: BLANK ROME LLP, WATERGATE, 600 NEW HAMPSHIRE AVENUE, N.W., WASHINGTON, DC, 20037, US

(21) Appl. No.: 11/794,464

(22) PCT Filed: Dec. 29, 2005
(86) PCT No.: PCT/US05/47221

§ 371 Date: Oct. 27, 2008

Related U.S. Application Data
(60) Provisional application No. 60/640,170, filed on Dec. 30, 2004.

(51) Int. Cl. A61K 38/10; G01N 33/566; G01N 33/68; A61K 38/17; A61K 38/08; C07K 14/47; A61P 35/00

(52) U.S. Cl. 514/14; 436/501; 436/86; 514/12; 514/16; 530/350

(57) ABSTRACT

The present invention relates to modulating, neutralizing, or inactivating the activity of migration inhibitory factor (MIF). In particularm, the present invention provides methods for modulating, neutralizing, or inactivating MIF activity by complexing MIF with other molecules, preferably a protein, that is capable of associating with MIF. These molecules can be used for treating diseases by the regulation of MIF activity.

This application claims priority to U.S. Provisional Patent Application No. 60/640,170, filed Dec. 30, 2004, which is incorporated herein by reference.

### FIELD OF THE INVENTION

The present invention relates to modulating, neutralizing, or inactivating the activity of migration inhibitory factor (MIF). In particular, the present invention relates to methods of treating inflammatory diseases by the regulation of MIF activity.

### BACKGROUND

MIF was first described thirty years ago and was designated as a cytokine, a chemical mediator, which regulates cell growth by inducing the expression of specific target genes. The initial described function of MIF was as a regulator of inflammation and immunity. It is expressed in the brain, and eye lens, is a delayed early response gene in fibroblasts, and it has been reported that this protein can be found in prostate tissues. MIF has been shown to be a pituitary, as well as macrophage cytokine and a critical mediator of septic shock. Recent studies also suggest that MIF may have an autocrine function for embryo development and is produced by the Leydig cells of the testes. Thus, it appears that this cytokine may play a fundamental role in cell growth regulation and possibly development.

MIF is a regulator of inflammation and innate, as well as adaptive, immune responses. However, current research suggests an even greater role for MIF, as it is present in a variety of immune and non-immune cells laugh et al., Crit. Care Med. 30: Suppl. S27-S35, 2002). MIF is constitutively expressed in tissues such as the anterior pituitary, prostate and bladder epithelia (Meyer-Siegler et al., Diag. Mol. Path. 7:44-50, 1998; Meyer-Siegler, Cytokine 12:914-921, 2000; and Vera et al., BMC Neurosci. 4:17, 2003), gastric, small intestinal and colonic epithelia (Maaser et al., Gastroenterology 122:667-680, 2002), neuronal and non-neuronal cells in the brain (Bacher et al., Mol. Med. 4:217-230, 1998). Additionally, MIF is found in normal amounts in circulating blood and urine, suggesting that MIF activity is regulated, perhaps by interaction with other proteins.

As a proinflammatory cytokine, MIF counter-regulates the effects of glucocorticoids (Baugh et al., Crit. Care Med. 30: Suppl. S27-S35, 2002; and Lue et al., Microbes and Infection 4:449-460, 2002). Therefore, MIF has been proposed to play a critical role in immune and inflammatory diseases including septic shock (Bernhagen et al., Nature 365:756-793, 1993), rheumatoid arthritis (Leech et al., Arthritis & Rheumatism 42:1601-1608, 1999), delayed-type hypersensitivity (Brown et al., Transplantation 71:1777-1783, 2001), Crohn's disease (De Jong et al., Nature Immunology 2:1061-1066, 2001), gastric ulcer formation (Huang et al., Gastroenterology 121:619-630, 2001), and prostate cancer (Meyer-Siegler et al., Diag. Mol. Path. 7:44-50, 1998; Meyer-Siegler, Cytokine 12:914-921, 2000; and Meyer-Siegler et al., BMC Cancer 5:73, 2005). Treatment with anti-MIF antibodies has been reported to prevent experimental colitis and treat established colitis in experimental animals (De Jong et al., Nature Immunology 2:1061-1066, 2001) and reduce experimental bladder inflammation in rats (Meyer-Siegler et al., J. Urol. 172:504-509, 2004). Therefore, regulation of MIF represents a useful therapeutic tool in the treatment of different inflammatory conditions.

U.S. Pat. No. 6,774,227 to Bucala et al., the disclosure of which is incorporated herein by reference, discloses methods and compositions for treading disorders related to cellular overproliferation by neutralizing the production or activity of MIF. Buccala et al. disclose methods including the use of factors which bind to MIF and neutralize its biological activity; the use of MIF-receptor antagonists; the use of factors that inhibit the enzymatic activity of MIF; the use of compounds that inhibit the release of MIF from cellular sources in the body; and the use of nucleotide sequences derived from MIF coding, non-coding, and/or regulatory sequences to prevent or reduce MIF expression.

U.S. Pat. No. 6,645,493 to Bucala et al., the disclosure of which is incorporated herein by reference, discloses anti-MIF antibodies for inhibiting the release and/or biological activity of MIF. The antibodies are used to treat various conditions involving cytokine-mediated toxicity, which includes, shock, inflammation, graft versus host disease, and/or autoimmune diseases.

U.S. Pat. No. 6,599,938 to Al-Abed et al., the disclosure of which is incorporated herein by reference, discloses a certain Schiff base condensation products having MIF antagonist activity. These compounds are useful in treating a variety of diseases involving inflammatory activity or pro-inflammatory cytokine responses, such as autoimmune diseases, asthma, arthritis, EAE, ARDS and various forms of sepsis and septic shock, and other conditions characterized by underlying MIF responses including, for instance, tumor growth and neovascularization.

These patents use compounds that are not normally associated with MIF, thus, may result in unwanted side effects and complications when used in vivo. Therefore, there remains a need for inhibition of MIF that utilizes molecules that are naturally produced by the host and causes minimal side effects.

### SUMMARY OF THE INVENTION

Applicant has discovered that 1) MIF dos not exist by itself as an individual protein in urine, blood, or tissues; 2) MIF is associated with acute phase proteins in vivo; 3) the association of MIF with the acute phase proteins or interaction of small molecules with MIF binding domain affect MIF activity, including the expression of other factors regulated by MIF, cell proliferation, and induction of apoptosis.

Based on the discoveries, the present invention provides methods for modulating, neutralizing, or inactivating MIF activity by complexing MIF with other molecules. The methods comprise contacting MIF with a molecule, preferably a protein that is capable of associating with MIF.

The present invention further provides methods for treating or ameliorating diseases associated with overproliferation of MIF by complexing MIF with other molecules. The methods comprise administering a molecule, preferably a protein, to an individual suffering from the inflammatory disease, wherein the molecule is capable of associating with MIF. These diseases include, but are not limited to, certain cancers (prostate and bladder), inflammatory, and proinflammatory diseases.

The present invention further provides methods for screening for compounds that encourage association of MIF with other molecules. The methods comprise 1) contacting MIF with a molecule, preferably a protein, that is capable of associating with MIF to form a complex; 2) contacting a candidate compound with the complex; 3) determining the degree of complex formation; and 4) determining whether the candidate compound encourages association of MIF with the molecule by comparing the degree of complex formation in step 3) with the degree of complex formation before the addition of the candidate compound.

The present invention further provides pharmaceutical compositions for the treatment of diseases associated with overproliferation of MIF or MIF activity. The compositions comprise a therapeutically effective amount of a molecule, preferably a protein that is capable of associating with MIF.

In a preferred embodiment of the present invention, the molecule capable of associating with MIF is α-1-inhibitor-3, α-1-inhibitor 3 precursor, α-2 macroglobulin, complement component 4a, α-1 macroglobulin, ceruloplasmin precursor, ceruloplasmin, uromodulin, or combinations thereof. Most preferably, the ceruloplasmin has an amino acid sequence containing at least SEQ ID NO:1 (QSEDSTFYLGER); and the uromodulin has an amino acid sequence containing at least SEQ ID NO:2 (YFIIQDR). These molecules are acute phase proteins, whose concentrations change during the inflammatory response (Ceciliani et al., Protein Pept Lett 9:211-223, 2002, which is incorporated herein by reference). These proteins function as generalized proteinase inhibitors, cytokine carriers and/or inhibitors; and can usually be purified from natural sources or made recombinantly if the protein or DNA sequence is known.

### BRIEF DESCRIPTION OF THE DRAWINGS

FIG. 1 shows a MIF Western blot of intraluminal fluid (defined as fluid contained within the bladder) following treatment with substance P.

FIG. 2 shows a MIF Western blot of intraluminal fluid following treatment with capsaicin.

FIG. 3 shows a MIF Western blot of bladder homogenates following treatment with substance P or capsaicin.

FIG. 4 shows Coomassie stain and MIF Western blot of intraluminal fluid

FIG. 5 shows immunoprecipitation of intraluminal fluid with MIF antibody and detection of SDS-PAGE separated proteins using α-1-I-3 antibody.

FIG. 6 shows MIF in urine samples obtained following routine urinalysis.

FIG. 7 shows Coomassie stain and MIF Western blot identifying urine MIF complex proteins in urine.

FIG. 8 shows co-immunoprecipitation of urine proteins A) immunoprecipitation of MIF; and B) immunoprecipitation of α-2 macroglobulin.

FIG. 9 compares urine MIF amounts in non-UTI and UTI patients determined by A) ELISA; and B) Western blot.

### DETAILED DESCRIPTION OF THE PREFERRED EMBODIMENT

The regulation, neutralization, or inhibition of MIF in accordance with the invention can be accomplished in a number of ways, which may include, but are not limited to, the use of factors which bind to MIF and neutralize its biological activity; or the use of MIF-receptor antagonists. Any of the foregoing may be utilized individually or in combination to inhibit MIF activity in the treatment of conditions related to cellular overproliferation, and further may be combined with any other antitumor therapy, such as pharmacological, surgical, cytokine, steroids or gene therapy, or any combination thereof. Preferably, the regulation, neutralization, or inhibition is accomplished by binding MIF to molecules, preferably proteins that associates with MIF. In a more preferred embodiment, the proteins are those normally associated with MIF in the urine, blood, or tissues. More preferably, these proteins can be, but are not limited to, α1-inhibitor 3, α-1-inhibitor 3 precursor, α-2 macroglobulin, complement component 4a, α-1 macroglobulin, cerulaplasmin precursor, or combinations thereof. Most preferably, the ceruloplasmin has an amino acid sequence containing at least SEQ ID NO:1 (QSEDSTFYLGER); and the uromodulin has an amino acid sequence containing at least SEQ ID NO:2 (YFIIQDR). “Association” and “associated with,” as used herein, refers to various processes of protein combination or protein binding. The combination or binding can be relatively weak chemical bonding, such as hydration, solvation, and complex formation, or strong chemical bonding, such as covalent bonding (including disulfide bonds), ionic bonding, and hydrogen bonding.

The present invention further provides methods for treating or ameliorating diseases associated with overproliferation of MIF by complexing MIF with other molecules. The methods comprise administering a molecule, preferably a protein, to an individual suffering from the inflammatory disease, wherein the molecule is capable of associating with MIF and is effective in regulating, neutralizing, or inhibiting MIF function.

The molecule can be administered to the individual through various routes of administration, including, but is not limited to, topical, cutaneous, oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections, and optionally in a depot or sustained release formulation. In an embodiment, topical administration utilizes excipients such as creams, emulsifiers, and oils. Further embodiments, topical administration utilizes dermal absorption enhancers selected from the group consisting of dimethyl sulfoxide, menthol, lauryl alcohol, lauric acid, arachidonic acid and C₁₀-C₂₀ polyhydroxy acids and thymol.

For injection, the composition of the invention may be formulated in aqueous solutions, preferably in physiologically compatible buffers, such as Hank's solution, Ringer's solution, or physiological saline buffer. For transmucosal administration, penetrants appropriate to the barrier to be permeated are used in the formulation. Such penetrants are known in the art.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

For oral administration, the composition can be formulated readily by combining the molecules (capable of associating with MIF) with pharmaceutically acceptable carriers well known to those in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the compound with a solid excipient, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Formulations for other routes of administration are well known in the art and apparent to one of ordinary skill in the art.

An effective dose refers to that amount of the molecule (that associates with MIF) that results in a reduction in the development or severity of a disease characterized by over proliferation of MIF or MIF activity. Toxicity and therapeutic efficacy of such compounds can be determined by standard pharmaceutical, pharmacological, and toxicological procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and SD₅₀. Compounds that exhibit high therapeutic indices are preferred. The data obtained from cell culture assays or animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition.

Dosage amount and interval may be adjusted individually to provide plasma levels of the molecule which are sufficient to maintain the desired modulating, neutralizing, or inactivating effects on MIF activity, or minimal effective concentration (MEC). The MEC will vary for each compound but can be estimated from in vitro data; e.g., the concentration necessary to achieve a 50-90% inhibition of MIF activity. Dosages necessary to achieve the MEC will depend on individual characteristics and route of administration. However, HPLC assays, bioassays or immunoassays can be used to determine plasma concentrations.

Dosage intervals can also be determined using the MEC value. Compounds should be administered using a regimen that maintains plasma levels above the MEC for 10-90% of the time, preferably between 30-90% and most preferably between 50-90%.

In another embodiment, the present invention further provides methods for screening for compounds that encourage association of MIF with other molecules. The methods comprise 1) contacting MIF with a molecule, preferably a protein, that is capable of associating with MIF to form a complex; 2) contacting a candidate compound with the complex; 3) determining the degree of complex formation; and 4) determining whether the candidate compound encourages association of MIF with the molecule by comparing the degree of complex formation in step 3) with the degree of complex formation before the addition of the candidate compound. If the candidate compound is found to encourage or discourage the association of MIF with the molecule capable of associating with MIF, the candidate compound may well be a drug candidate that is effective in treating or ameliorating diseases associated with overproduction of MIF.

The candidate compound can be, but are not limited to, peptides, small molecules, vitamin derivatives, as well as carbohydrates. Dominant negative proteins, DNA encoding these proteins, antibodies to these proteins, peptide fragments of these proteins or mimics of these proteins may be introduced into the patient to affect function. “Mimic” as used herein refers to the modification of a region or several regions of a peptide molecule to provide a structure chemically different from the parent peptide but topographically and functionally similar to the parent peptide (see Grant (1995), in Molecular Biology and Biotechnology, Meyers (editor) VCH Publishers). A skilled artisan can readily recognize that there is no limit as to the structural nature of the candidate compound.

Typically, the candidate compound effect on the binding of MIF and its associated molecule is observed. In its simplest form, MIF and its associated molecule are mixed with the candidate compound in solution. The degree of binding of MIF and its associated molecule with and without the candidate compound are the compared to determine if the candidate molecule has an effect on the binding. If the candidate compound affects the binding of MIF with its associated molecule, the compound is further tested for its potential use as a regulator of MIF activity.

The compound screening can be accomplished individually; however, it is preferred that high-throughput screening be applied to screen large numbers of candidates in a short period of time. Preferably, hundreds, thousands, or tens of thousands of candidate compounds can be tested simultaneously for their effect of the binding of MIF with a compound capable of associating with MIF. High-throughput screening can be accomplished with current, sophisticated techniques using microarray and/or microfluidic systems. One possible high-throughput screening method is disclosed in U.S. Pat. Nos. 6,630,353 to Parce et al., U.S. Pat. No. 6,613,580 to Chow et al., the disclosures of which are incorporated herein by reference.

Without further description, it is believed that one of ordinary skill in the art can, using the preceding description and the following illustrative examples, make and utilize the compounds of the present invention and practice the claimed methods. The following example is given to illustrate the present invention. It should be understood that the invention is not to be limited to the specific conditions or details described in this example.

### EXAMPLE 1

### MIF is Only Found Complexed With Other Proteins in vivo

In order to establish the form of MIF found in tissues and the changes associated with treatments designed to induce neurogenic inflammation; rat intraluminal fluid was analyzed for MIF using ELISA (Table 1). As reported previously, substance P (SP) treatment induced a significant increase in intraluminal MIF amounts (Table 1). Intravesical capsaicin resulted in a dose-dependent excretion of MIF into the intraluminal fluid (Table 1).

TABLE 1
Changes in intraluminal MIF concentrations

| Inflammatory inducer | Intraluminal MIF (ng/mL) | |
|---|---|---|
| | Control | Treatment |
| | (n = 5) | (n = 5) |
| Substance P | 1.8 ± 0.7 | 9.5 ± 1.0*** |
| (subcutaneous) | | |

| | Control | 0.1 mM | 1.0 mM |
|---|---|---|---|
| | (n = 3) | (n = 3) | (n = 3) |
| Capsaicin | 4.1 ± 0.6 | 13.9 ± 3.2 | 28.6 ± 3.8** |
| (intravesical) | | | |

Intraluminal MIF amounts were determined using a rat MIF specific ELISA. Data are expressed as mean MIF ng/mL of intraluminal fluid ± S.E.M.,
**= p < 0.01,
***= p < 0.001.

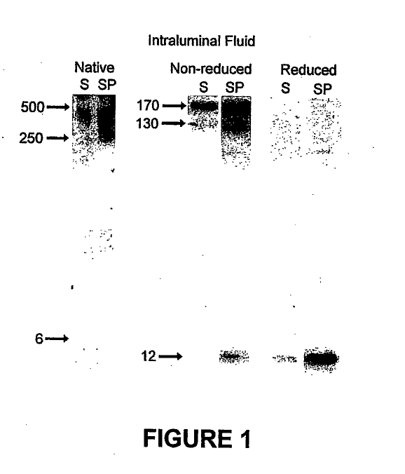

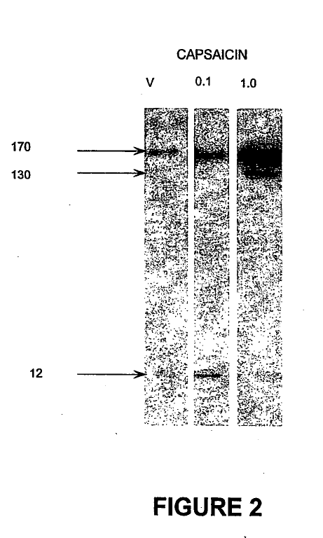

Rat urine, intraluminal fluid and bladder tissue were also analyzed by native, non-reducing and reducing polyacrylamide gel electrophoresis (FIGS. 1 and 2). Intraluminal fluid separated under native conditions contained a broad MIF immunoreactive band ranging from 250 to 500 kDa (FIG. 1, native). Non-reducing conditions of the same samples from saline treated animals resulted in two bands of 170 kDa and 130 kDa with SP treatment resulting in an increase in the 130 kDa band (p<0.01). Very small amounts of a 12 kDa band (corresponding to monomer MIF) were detected in some saline control animals (FIG. 1, Non-reduced). Western blot analysis of the same samples run under reducing conditions detected only a 12 kDa MIP band. SP treatment resulted in an increase in 12 kDa MIF amounts that could be detected in intraluminal fluid separated under reducing conditions (FIG. 1, Reduced, p<0.001). Western blot data shown are representative of all experimental animals.

Intravesical capsaicin treatment resulted in a significant increase in the intraluminal amounts of the 170 kDa band (FIG. 2, p<0.005). Again the Western blot data shown are representative of all experimental animals.

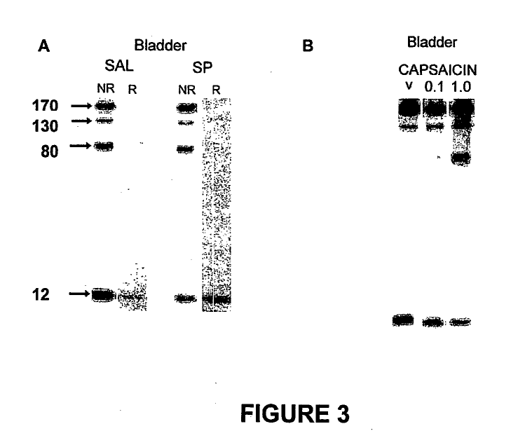

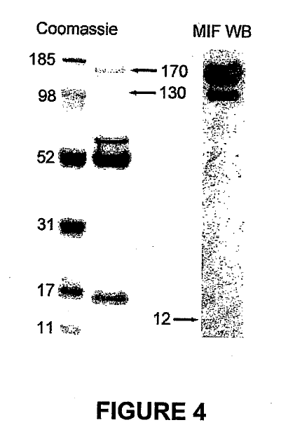

Bladder tissue homogenates from SP treated animals were separated by non-reducing SDS-PAGE and MIF reactive bands detected by Western blotting. In the bladder four MIF reactive bands are identified (170 kDa, 130 kDa, 80 kDa and 12 kDa). The 80 kDa band is exclusive to the bladder tissue homogenates and was seen in both control, SP treated and capsaicin treated bladders (FIG. 3). SP and capsaicin treatment resulted in a significant decrease in 12 kDa band found in bladder tissue. The identity of these bands has not been verified by immunoprecipitation. However, based upon the pattern it is likely that these bands are similar to those found in the intraluminal fluid. Additionally the 80 kDa band is unique to bladder tissue and may represent a different MIF inhibitor and/or binding molecule. In the bladder tissue 12 kDa MIF was detected under non-reducing and reducing conditions, indicating that a significant MIF amount is dissociated from the complexes by denaturing conditions. Under reducing conditions only 12 kDa MIF is detected, suggesting that the proteins in these complexes are associated via disulfide bonds.

In summary, native gel electrophoresis and Western blot analysis of intraluminal fluid and bladder tissue failed to identify monomer, dimer or trimer MIF, thus determining that MIF is predominantly associated with other proteins. MIF monomer was only identified following treatment with reducing agents, indicating that MIF is associated with other proteins via disulfide bonds.

### EXAMPLE 2

### Identification of MIF Associated Proteins as Alpha-1-Inhibitor-3

### Mass Spectroscopy

Duplicate samples of intraluminal fluid were separated by non-reducing NuPAGE electrophoresis with one sample analyzed by Western blotting and its duplicate Coomassie stained. X-ray film of the Western blot was used to identify MIF complex Coomassie stained bands for mass spectroscopy analysis (protein Chemistry Core Laboratory; University of Florida, Gainesville, Fla.). These bands were identified as several acute phase proteins (Table 2). The highest and most significant match (for either the 170 or 130 kDa band) was alpha-1-inhibitor-3 (α-1-I-3), however, peptides matching alpha-2-macroglobulin, complement component 4, and ceruloplasmin precursor were also identified.

TABLE 2
Proteins identified by mass spectroscopy

| | Reference | | | Peptides |
|---|---|---|---|---|
| Band | Number | Protein (molecular weight) | Score | Matched |
| 170 kDa | gi\|12831225 | α(1)-inhibitor 3 (166 kDa) | 501 | 20 |
| | gi\|112893 | α(1)-inhibitor 3 precursor (165 kDa) | 487 | 17 |
| | gi\|109550 | α-2-macroglobulin | 166 | 9 |
| | gi\|29789265 | Complement component 4a (194 kDa) | 114 | 5 |
| | gi\|202857 | α-1-macroglobulin (168 kDa) | 63 | 3 |
| 130 kDa | gi\|112893 | α(1)-inhibitor 3 precursor (165 kDa) | 121 | 5 |
| | gi\|2506226 | Ceruloplasmin precursor (121 kDa) | 104 | 9 |

### Verification of Identity of MIF Associated Proteins

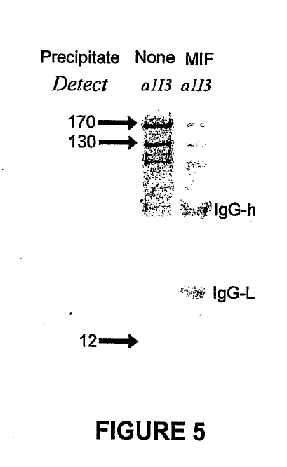

The interaction of MIF and α-1-I-3 was confirmed by immunoprecipitation with anti-MIF antibody (R&D Systems) followed by detection of α-1-I-3 by Western blotting (FIG. 5). Immunoprecipitation of intraluminal fluid from Substance P treated animals was accomplished with anti-MIF (5.0 μg/ml, AB-289-PB, R&D Systems) using Protein G agarose. Resultant protein complexes were separated on NuPAGE using reducing conditions and α-1-I-3 detected on the blots using a polygonal rabbit anti-rat antibody provided by Dr. G. A. Kaysen and was a gift from Dr. H. van Goor (Netherlands). This study verified that MIF was bound to α-1-I-3 (FIG. 5).

### EXAMPLE 3

### MIF Complexes in Human Urine

### Bacterial Cystitis Induced MIF Complexes

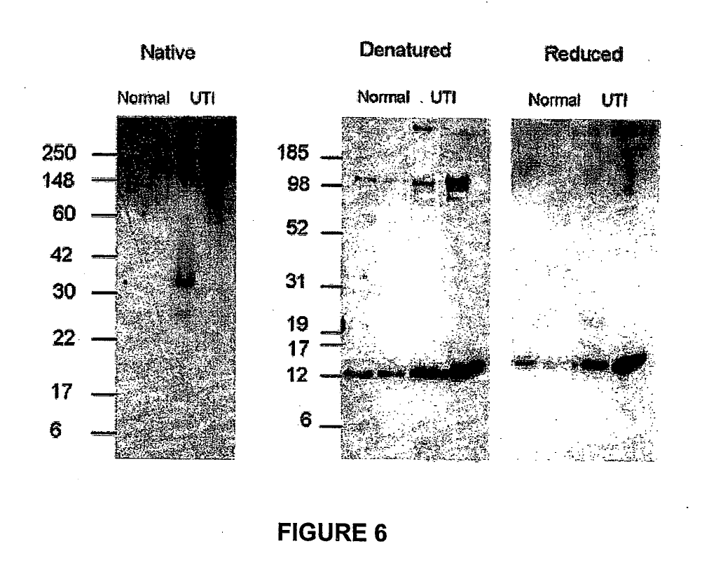

MIF western blot (WB) of urine proteins from UTI patients separated under native conditions resulted in a prominent high molecular weight band in the range of 150 to 500 kDa (FIG. 6). A less intense band at the same molecular weight range was observed in WB of urine from non-UTI patients. Under native WB conditions, a 12 kDa band indicative of monomeric MIF was not detected (FIG. 6). However, under denaturing conditions, WB of the same sample resulted in three prominent high molecular weight bands of 165, 135 and 100 kDa as well as the 12 kDa monomeric MIF band (FIG. 6). MIF staining intensity was greater in the samples from UTI patients. Under reducing conditions only a single MIF staining band at 12 kDa was evident in all the urine samples. The MIF band staining intensity was greatest in urine from UTI patients (FIG. 6).

Thus, in agreement with Examples 1-2, MIF in human urine is found in high molecular weight complexes. In all urine samples analyzed, a significant portion (65.7±4.5%) of monomeric MIF (12 kDa) interacts with other molecules via non-covalent bonds since denaturing conditions revealed a prominent 12 kDa MIF band. Finally, reducing conditions showed that the higher molecular weight bands observed in the urine (165, 135, 100 kDa) interact with MIF through covalent bonds since the complex was disrupted only in conditions that allow dissociation of disulfide bonds.

### Identification of Proteins Complexed With MIF

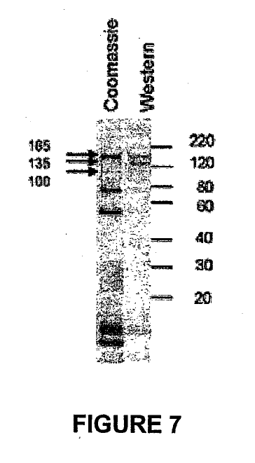

Denaturing PAGE MIF WB analysis of human urine revealed three prominent high molecular weight bands at 165, 135 and 100 kDa (FIGS. 6 and 7A). These high molecular weight bands were excised from Coomassie stained gels, analyzed by mass spectroscopy and identified as the acute phase proteins ceruloplasmin (165 kDa) and albumin (100 kDa), as well as the predominant urine protein uromodulin (72 kDa) (Table 3).

TABLE 3
Proteins identified by mass spectroscopy

| | Reference | | | Peptides |
|---|---|---|---|---|
| Band | Number | Protein (molecular weight) | Score | Matched |
| 165 kDa | gi\|180249 | Ceruloplasmin (98 kDa) | 121 | 4 |
| 135 kDa | gi\|4507833 | Uromodulin (72 KDa) | 135 | 12 |
| 100 kDa | gi\|28592 | Serum albumin (71 kDa) | 1063 | 26 |

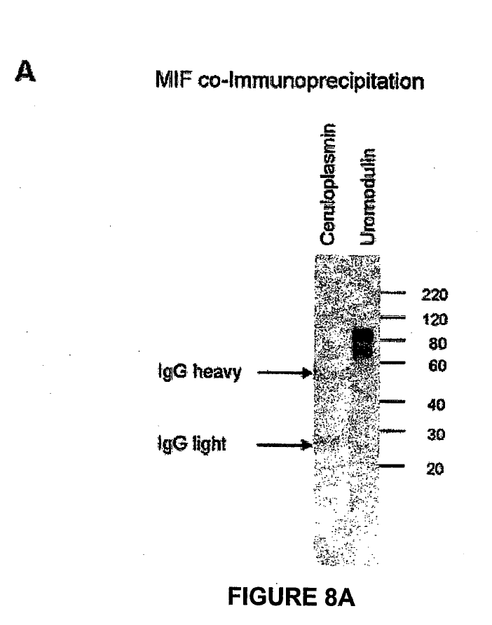

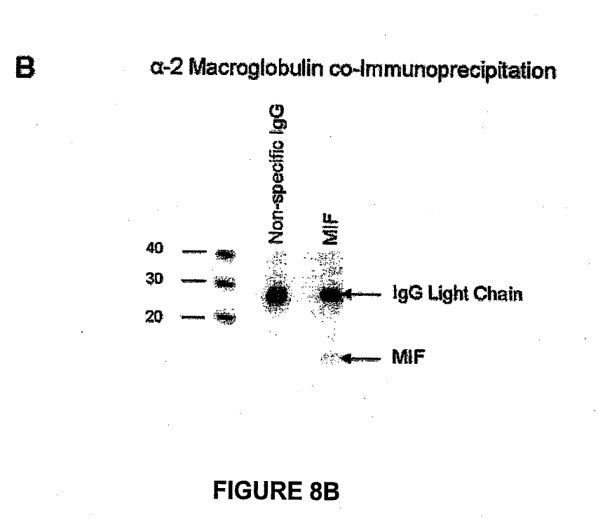

Co-immunoprecipitation studies using anti-MIF followed by WB using antisera to proteins identified by mass spectroscopy confirmed the interaction of MIF with ceruloplasmin and uromodulin (FIG. 8A). MIF immunoprecipitation with ceruloplasmin WB analysis identified two bands of 135 and 64 kDa, while uromodulin WB of MIF immunoprecipitation revealed an intense band at 86 kDa.

We had expected to identify MIF interaction with proteins in the α-2-macroglobulin superfamily since previously we identified interaction between MIF and α-1-inhibitor-3, a rodent specific α-2-macroglobulin superfamily protein. α-2-macroglubulin WB of proteins co-immunoprecipitated with MIF failed to show α-2-macroglobulin. However, MIF WB of α-2-macroglobulin precipitated protein identified a faint 12-kDa band (FIG. 8B) indicating that these two proteins interact in human urine.

### Comparison of MIF Urine Amounts in Patients With and Without Bacterial Cystitis

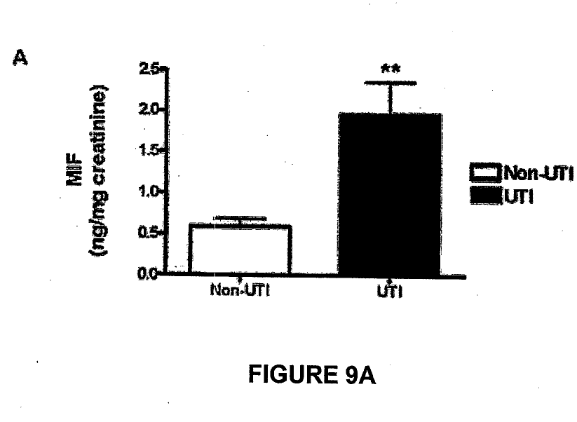

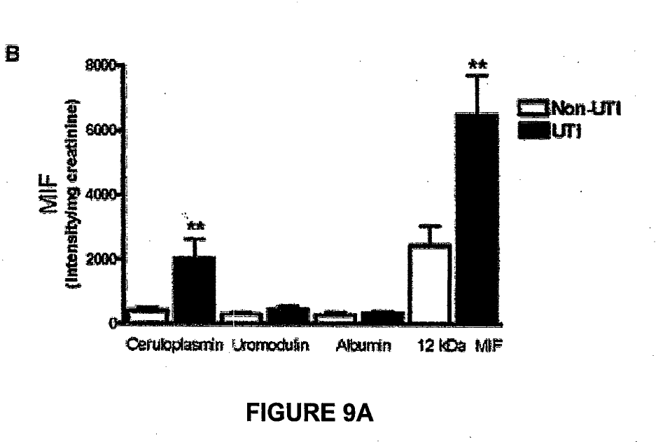

As seen in FIG. 9A, ELISA detected significantly increased MIF amounts in urine of UTI patients (1.96±0.40 ng/mg creatinine) compared to non-UTI patients (0.59±0.09 ng/mg creatinine, p<0.01). For all samples analyzed, creatinine ranges were within reported normal values (10-300 mg/dl) with non-UTI group mean creatinine equal to 115.0±21.27 mg/dl and the UTI group mean creatinine 110.3±8.46 mg/dl. In addition, analysis of changes in specific bands in WB under denaturing conditions showed that UTI urines exhibited a significant increase in both the ceruloplasmin (FIG. 9B, p<0.01) and the 12 kDa MIF monomeric band (FIG. 9B, p<0.01).

The invention has been disclosed broadly and illustrated in reference to representative embodiments described above. Those skilled in the art will recognize that various modifications can be made to the present invention without departing from the spirit and scope thereof.

What is claimed is:

1. A method for modulating, neutralizing, or inactivating MIF activity comprising the step of contacting MIF with a molecule selected from the group consisting of is α-1-inhibitor-3, α-1-inhibitor 3 precursor, α-2 macroglobulin, complement component 4a, α-1 macroglobulin, ceruloplasmin precursor, ceruloplasmin, uromodulin, and combinations thereof.

2. The method of claim 1, wherein the ceruloplasmin has an amino acid sequence comprising SEQ ID NO:1.

3. The method of claim 1, wherein the uromodulin has an amino acid sequence comprising SEQ ID NO:2.

4. A method for treating or ameliorating diseases associated with overproliferation of MIF comprising the step of administering to an individual having the disease a molecule selected from the group consisting of α-1-inhibitor-3, α-1-inhibitor 3 precursor, α-2 macroglobulin, complement component 4a, α-1 macroglobulin, ceruloplasmin precursor, ceruloplasmin, uromodulin, and combinations thereof.

5. The method of claim 4, wherein the ceruloplasmin has an amino acid sequence comprising SEQ ID NO:1.

6. The method of claim 4, wherein the uromodulin has an amino acid sequence comprising SEQ ID NO:2.

7. The method of claim 4, wherein the disease is selected from the group consisting of cancer, inflammatory disease, and proinflammatory disease.

8. The method of claim 7, wherein the cancer is prostate cancer or bladder cancer.

9. A method for screening for compounds that affect association of MIF with other molecules, the method comprising the steps of
a) contacting MIF with a molecule capable of associating with MIF to form a complex;
b) contacting a candidate compound with the complex;
c) determining the degree of complex formation; and
d) determining whether the candidate compound affect the association of MIF with the molecule by comparing the degree of complex formation in step c) with a degree of complex formation before the addition of the candidate compound.

10. The method of claim 9, wherein the molecule capable of associating with MIF is selected from the group consisting of α-1-inhibitor-3, α-1 -inhibitor 3 precursor, α-2 macroglobulin, complement component 4a, α-1 macroglobulin, ceruloplasmin precursor, ceruloplasmin, uromodulin, and combinations thereof.

11. The method of claim 9, wherein the ceruloplasmin has an amino acid sequence comprising SEQ ID NO:1.

12. The method of claim 9, wherein the uromodulin has an amino acid sequence comprising SEQ ID NO:2.

13. The method of claim 9, wherein the degree of complex formation is the molar ratio of the complex to free MIF.

14. The method of claim 9, Wherein the candidate compound is selected from the group consisting of peptides, small molecules, vitamin derivatives, and carbohydrates.


SEQUENCE LISTING (2 sequences)

SEQ ID NO: 1 - protein, 12 aa, rat
QSEDSTFYLGER

SEQ ID NO: 2 - protein, 7 aa, Homo sapiens
YFIIQDR